(19) United States
(12) Patent Application Publication
(10) Pub. No.: US 2024/0415944 A1
(43) Pub. Date: Dec. 19, 2024

(54) Aqueous aprotinin-containing antiviral pharmaceutical composition

(71) Applicants: Andrey Alexandrovich IVASHCHENKO, Moscow (RU); Nikolay Filippovich SAVCHUK, Rancho Santa Fe, CA (US); Alena Alexandrovna IVACHTCHENKO, Hallandale, FL (US); Alexandre Vasilievich IVACHTCHENKO, Hallandale, FL (US); ALLA CHEM, LLC, Hallandale, FL (US)

(72) Inventors: Alexandre Vasilievich Ivachtchenko, Hallandale, FL (US); Andrey Alexandrovich Ivashchenko, Moscow (RU); Nikolay Filippovich Savchuk, Rancho Santa Fe, CA (US); Alena Alexandrovna Ivachtchenko, Hallandale, FL (US)

(21) Appl. No.: 17/298,958

(22) PCT Filed: Apr. 6, 2021
(86) PCT No.: PCT/RU2021/000145

§ 371 Date: Jun. 2, 2021

(51) Int. Cl. A61K 38/57; A61K 9/08; A61M 11/00; A61P 31/14; A61P 31/16

(52) U.S. Cl. CPC A61K 38/57; A61K 9/08; A61M 11/00; A61P 31/14; A61P 31/16

(57) ABSTRACT

The invention relates to a new aqueous aprotinin-containing antiviral pharmaceutical composition with aprotinin activity from 50 KIU/ml to 950 KIU/ml, optionally containing excipients, for the prevention and treatment of acute respiratory viral infections (ARVI) (acute respiratory infection—ARI). The invention also relates to a new drug for the treatment and prevention of ARVI, comprising said composition in a form suitable for inhalation, nasal or drip use. The medicament can be a nasal spray, or a throat and mouth spray, placed in a vial or vial with a metering valve for a spray or with a dropper dispenser. The medicament can also be aqueous aprotinin-containing antiviral pharmaceutical composition, in a form suitable for inhalation, placed in a vial or vial, container or ampoule for inhalation by a nebulizer. The invention also relates to the use of VASPFC or a drug based on it for the prevention and treatment of ARVI. In particular, for the prevention and treatment of acute respiratory viral infections in patients infected with influenza viruses or coronaviruses, including SARS-CoV-2, as well as for the prevention and treatment of influenza pneumonia and coronavirus disease COVID-19.

This invention relates to a novel aqueous aprotinin-containing antiviral pharmaceutical composition (AACAPC), which optionally contains excipients and is intended for the prevention and treatment of acute respiratory viral infections (ARVI). ARVI is a group of clinically and morphologically similar acute inflammatory respiratory diseases, the causative agents of which are usually viruses with tropism for the respiratory epithelium. ARVI is the most common group of diseases in the world comprising more than 200 varieties of diseases, of which the best known is influenza. In addition to influenza, ARVIs include respiratory syncytial, rhinovirus, coronavirus, adenovirus, and other infections that cause catarrhal inflammation of the respiratory tract.

Colds are commonly referred to as ARVI of a mild course affecting mainly the nasal passages. In most cases, respiratory viral infections occur as a mild disease and go away by themselves, as the immune system eventually cleanses the body of the virus on its own.

Severe cases occur when viruses enter the lower respiratory tract resulting, in addition to bronchitis, in viral pneumonia, acute respiratory distress syndrome or severe acute respiratory syndrome (SARS or COVID-19). Also, viral respiratory infections can lead to exacerbation of chronic diseases [M. C. Savenkova. Influenza and other acute respiratory infections in children. Aspects of antiviral therapy: https://cyberleninka.ru/article/n/gripp-i-drugie-ostrye-respiratornye-virusnye-infektsii-u-detey-aspekty-protivovirusnoy-terapii/viewer. Chuchalin A. G., Avdeev S. N., Chernyaev A. L. et al. Federal clinical guidelines of the Russian Respiratory Society for the diagnosis and treatment of severe forms of influenza. Pulmonologiya 2014, 5, 11-19. https://doi.org/10.18093/0869-0189-2014-0-5-11-19].

According to WHO data, the annual incidence of influenza is 5-10%, with significant morbidity and mortality, especially among at-risk groups. The socioeconomic consequences are also significant. Most influenza drugs that are currently on the market are neuraminidase inhibitors (zanamivir, oseltamivir, perimivir) or M2 protein forming proton ion channels (amantadine, rimantadine) [Hayden, F. G. Antivirals for influenza: historical perspectives and lessons learned. Antiviral Res 71, 372-8 (2006)].

Until recently, however, neuraminidase inhibitors such as oseltamivir were the only available anti-influenza drugs that complement the preventive effect of vaccination [A Hsieh, T Fumeaux. Quel rapport efficacité/effets secondaires pour l'oseltamivir dans le traitement de la grippe chez l'adulte? Rev Med Suisse 2015, 11. 576-577. https://www.revmed.ch/RMS/2015/RMS-N-464/Quel-rapport-efficacite-effets-secondaires-pour-l-oseltamivir-dans-le-traitement-de-la-grippe-chez-l-adulte]. Note that since its launch in 1999, Roche has sold more than $18 billion worth of oseltamivir [Dr. Peter. Doctors around the world are up against a flu drug. Why, after 20 years of use, Cochrane's expert “sued” Tamiflu: https://doctorpiter-ru.turbopages.org/doctorpiter.ru/s/articles/27999/]. Oseltamivir in adult influenza patients accelerates time to relief of clinical symptoms by 17.8-25.2 hours (mean time to relief was 97.5 hours for oseltamivir and 122.7 hours for placebo groups), reduces the risk of lower respiratory complications and hospitalization but significantly increases the frequency of nausea and vomiting. It increases the risk of nausea (RR 1.60) and vomiting (RR 2.43) [J Dobson, R J Whitley, S Pocock, Arnold S Monto. Oseltamivir treatment for influenza an adults: A meta-analysis of randomised controlled trials. Lancet 2015, 385(9979), 1729-1737. doi: 10.1016/S0140-6736(14)62449-1.]

Relatively recently, highly effective anti-influenza drugs, Sar-dependent endonuclease inhibitors, have been patented. In this series of inhibitors, the most effective seems to be baloxavir marboxil (BXM, S-033188) sold under the name Xofluza® [WO 2016175224 A1; WO 2018030463 (priority 2016)], and AV5124 [Andrei A Ivashchenko, Oleg D Mitkin, Jeremy C Jones, et al. Synthesis, inhibitory activity and oral dosing formulation of AV5124, the structural analogue of influenza virus endonuclease inhibitor baloxavir. J Chemother Antimicrob 2020, dkaa524, https://doi.org/10.1093/jac/dkaa524. RU 2720305 priority 2019]. Therapy with these drugs should be started within 48 hours after the onset of flu symptoms. A single dose of the drug is recommended. The safety profile of baloxavir is comparable to that of neuraminidase inhibitors. The most common adverse reactions (≥1%) in clinical trials of acute uncomplicated influenza were diarrhea (3%), bronchitis (3%), nausea (2%), sinusitis (2%), and headache (1%). [https://www.xofluza-hcp.com/?c=cap-175bce8ee7a&gclid=622a8ef14cf01b7cd9fbeec7c853f668&gclsrc=3p.ds&].

Separately it is worth mentioning the drug aprotinin, which has a broad-spectrum effect against acute respiratory infections [O. P. Zhirnov. A method of treatment of viral respiratory infections. RU 2054180 (1991), U.S. Pat. No. 5,723,439, JP 2774379B, EP 0563389. O. P. Zhirnov, A. V. Khanykov. Aerosol preparation based on aprotinin for the treatment of viral respiratory infections. (IN RUSSIAN). O. P. Zhirnov. Pharmaceutical aerosol composition of protease inhibitors with ozone-saving propellant and its preparation RU 2657523 (2011). O. Zhirnov. Combined aerosol composition based on protease inhibitors and its preparation. RU 2711080 (2015)].

Another known AACAPC is Antiprovir [Patent RU 2 738 885 C1, 2020] with aprotinin activity ≈9714 KIU/ml, which contains ≈1.36 mg of aprotinin in 1 ml of the composition and is intended for the prevention and treatment of severe acute viral respiratory infection (SARS-CoV-2) and coronavirus disease (COVID-19). Antiprovir contains 1.5 g of aprotinin, 85 g of sodium chloride, and 100 g of benzyl alcohol in 1.0 L of water for injection.

Aprotinin is a monomeric (single-chain) globular polypeptide derived from bovine organs, which has a molecular weight of 6512 and consists of 16 different types of amino acids arranged in a 58-long chain that folds into a stable compact tertiary structure of the “small SS-rich” type containing 3 disulfides, a twisted β-helices, and a C-terminal α-helices. It has been used in clinical practice since as early as the 1960s [A. M. Scheule et al. J. Thorac. Cardiovasc. Surg. 1999, 118(2), 348-353]. It is a polyvalent inhibitor of proteases (including plasmin, kininogenase, trypsin, chymotrypsin, kallikrein, including activating fibrinolysis) and exhibits antiviral activity, reduces blood fibrinolytic activity, inhibits fibrinolysis, and has a hemostatic effect in coagulopathies. Aprotinin activity depends on its quality and manufacturer and is expressed in kallikrein inactivating units (KIU). For example, aprotinin produced by Sigma-Aldrich (USA), which contains 98% of active ingredient, has an activity of 6500 KIU/mg [https://www.sigmaaldrich.com/catalog/search?term=A6103&interface=All&N=0&mode=partialmax&lang=en&region=US&focus=product&F=PR&ST=RS&N3=mode%20matchpartialmax&N5=All]; aprotinin in Gordox® has an activity of 7143 KIU/mg (100,000 KIU is 14 mg and 500,000 KIU is 70 mg of aprotinin) [https://apteka.ru/novosibirsk/preparation/gordoks/], and aprotinin produced by Wanhua Biochem (China) No. NATE-1892, GMP containing >95% of active ingredient has an >3.0 activity EPU/mg (>5400 KIU/mg) [https://www.creative-enzymes.com/product/aprotinin-from-bovine-recombinant_16436.html].

A number of pharmaceutical compositions containing aprotinin are known to be used for the therapy of various diseases.

In particular, a known pharmaceutical composition for the treatment of viral infections such as influenza, acute viral respiratory infections, and infectious inflammatory diseases of viral etiology containing, per 1 ml of the composition, aprotinin (6480 KIU/ml) of BBT Biotech GmbH (Germany), excipients at a rate of 0.23-0.30 mg (1490-1944 KIU/ml) including propellant: 1,1,1,1,2-tetrafluoroethane (0.70-0.84 ml/ml), solvents: ethanol (0.08-0.15 ml/ml), glycerol (0.05-0.10 ml/ml), and water (0.02-0.04 ml/ml), and stabilizer: peppermint oil (0.006-0.01 ml/ml). [Patent RU 2425691.https://www.vidal.ru/drugs/aerus_23575; https://www.rlsnet.ru/tn_index_id_44141.htm]. Based on this pharmaceutical composition, a safe, stable, and efficient AERUS® individual inhaler for the treatment of influenza and acute respiratory viral infections at early stages is produced [Poliklinika 4/2012; http://www.poliklin.ru/imagearticle/201204(1)/79-81.pdf; https://www.lvrach.ru/2014/01/15435873]. Aerus® is a metered-dose aerosol for inhalation, 85 KIU/dose, and is available in 35 000 KIU (350 doses) or 25 000 KIU (250 doses) in an aluminum can with a capacity of 30 or 20 ml, respectively, in a nasal or oral dosing device (valve) with safety caps. One dose volume of concentrated aprotinin solution (85 KIU) is 0.00043 ml (aprotinin activity 197 674 KIU/ml [https://kiberis.ru/?p=15408].

Also available are a number of other pharmaceutical aprotinin-based compositions including such brand names as Trasilol®, Gordox®, Aprotex®, Traskolan, etc. that are used for the prevention of intraoperative blood loss and reduction of hemotransfusion during coronary artery bypass graft operations using CPR in adult patients who are at high risk for bleeding or need in hemotransfusion [https://yandex.ru/health/pills/product/gordoks-203. https://medside.ru/trasilol]. The drug is also recommended as a prophylactic treatment for patients in whom an increased risk of bleeding or need for transfusion can be expected [https://www.vidal.by/poisk_preparatov/gordox.html].

Trasilol® [https://www.rlsnet.ru/tn_index_id_16535.htm#sostav-i-forma-vypuska], Gordox® [https://yandex.ru/health/pills/product/gordoks-203], Aprotex® [https://health.mail.ru/drug/aprotex_1/], Traskolan [https://kiberis.ru/?p=13303] are concentrated aqueous aprotinin solutions with aprotinin activity from 5000 KIU/ml to 10000 KIU/ml, containing excipients selected from the following series: sodium chloride, sodium hydroxide, 1M hydrochloric acid solution, benzyl alcohol, etc.

Listed below are definitions of various terms used throughout the specification of this invention.

The term “medicinal drug” refers to a compound (or a mixture of compounds forming a pharmaceutical composition) in tablets, capsules, injections, ointments, sprays, inhalation solutions, including nasal or drip solutions, or other finished dosage forms intended for the restoration, improvement, or modification of physiological functions in humans and animals as well as for the treatment and prophylaxis of diseases, for diagnostics, anesthesia, contraception, cosmetology, etc.

The term “pharmaceutical composition” refers to a composition comprising an active ingredient (substance), for instance, aprotinin, and at least one of the components selected from the group consisting of pharmaceutically acceptable and pharmacologically compatible fillers, solvents, diluents, carriers, excipients, distributing, and sensing agents, delivery agents such as preservatives, stabilizers, fillers, disintegrators, moisteners, emulsifiers, suspending agents, thickeners, sweeteners, flavoring agents, aromatizing agents, antibacterial agents, fungicides, lubricants, and prolonged delivery controllers, the choice and proportions of which depend on the nature and route of administration and dosage.

Examples of suitable suspending agents are ethoxylated isostearyl alcohol, polyoxyethylene, sorbitol and sorbitol ether, microcrystalline cellulose, aluminum metahydroxide, bentonite, agar-agar and tragacant, and mixtures thereof. Protection against microorganisms can be provided using various antibacterial and antifungal agents, such as parabens, chlorobutanol, sorbic acid, and the like. Said composition may also include isotonic agents, such as sugar, sodium chloride, and the like. Examples of suitable carriers, solvents, diluents and delivery agents include water, ethanol, polyalcohols and mixtures thereof, natural oils (such as olive oil), and so on.

A pharmaceutical composition for oral, sublingual, transdermal, intramuscular, intravenous, subcutaneous, and local or rectal administration of the active ingredient, alone or in combination with another active ingredient, may be administered to animals and people in a standard administration form as a mixture with traditional pharmaceutical carriers. Suitable standard administration forms include oral forms, such as tablets, gelatin capsules, pills, powders, granules, chewing gums, and oral solutions or suspensions; sublingual and transbuccal administration forms; aerosols; sprays, and other administration forms.

The term “inert filler” as used herein refers to a compound that is used for preparing a pharmaceutical composition and is, as a rule, safe, nontoxic, and neither biologically nor otherwise undesirable, and comprises excipients acceptable for veterinary and human pharmaceutical use. Compounds of this invention may be administered individually, but they will be generally administered in a mixture with one or more pharmaceutically acceptable excipients, diluents, or carriers chosen depending on the contemplated route of drug administration and standard pharmaceutical practice.

The term “therapeutically effective amount,” or a dose, as used herein, refers to an amount of a substance, prodrug, or drug needed for alleviating the symptoms of the disease in the subject. The dose of a substance, prodrug, or drug will meet individual demands in each particular case. Said dose may vary in a wide range depending on numerous factors like the severity of the disease to be treated, the age and the general condition of the patient, other medicaments used for the patient's treatment, the mode and route of administration, and the experience of the attending doctor. As a rule, in order to alleviate or eliminate the virus, a higher loading dose is given at the beginning of treatment with a subsequent reduction of the dose to a level sufficient to prevent an infection outburst.

The term “subject” refers to a mammal including, but not limited to, cattle, hogs, sheep, chickens, turkeys, buffalos, lamas, ostriches, dogs, cats, and humans; a human subject is most preferable. According to this invention, the method of treatment of the subject involves the use of aprotinin as an active ingredient (substance), optionally in combination with other active ingredients.

The main disadvantage of using the aforementioned AACAPCs is their high activity, usually between 5000 KIU/ml and 197674 KIU/ml aprotinin, leading to noticeable side effects such as arterial hypotension and/or tachycardia; allergic reactions (skin rash, urticaria, rhinitis, conjunctivitis, bronchospasm, myalgia, symptoms of anaphylactic reactions up to the development of anaphylactic shock (occurs more frequently after repeated infusions of aprotinin); psychotic reactions, hallucinations, mental confusion; rapid infusion may provoke nausea and/or vomiting; and prolonged infusion may lead to thrombophlebitis [https://www.rlsnet.ru/tn_index_id_44141.htm#pobochnye-dejstviya. https://www.vidal.by/pdf/trasylol-instrukciya-po-primeneniyu.pdf. https://www.vidal.by/pdf/trasylol-instrukciya-po-primeneniyu.pdf. https://www.rlsnet.ru/tn_index_id_44141.htm].

The purpose of the present invention is to reduce and/or eliminate the above-mentioned side effects of pharmaceutical compositions while maintaining high efficacy in the prevention and treatment of acute respiratory viral infections (ARVI).

The subject of the present invention is an aqueous aprotinin-containing antiviral pharmaceutical composition (AACAPC) with aprotinin activity from 50 KIU/ml to 950 KIU/ml for ARVI prevention and treatment.

The proposed AACAPC may contain excipients, such as sodium chloryl, benzyl alcohol, sodium hydroxide, lactose, propellant (e.g., 1.1,1,1,2-tetrafluoroethane or 1,1,1,1,2,3,3,3-heptafluoropropane), and others.

A further subject of this infection is a medicinal drug for AVRI prevention and treatment. The medicinal drug contains AACAPC with aprotinin activity from 50 KIU/ml to 950 KIU/ml suitable for inhalation, nasal, or drip use.

The medicinal drug containing said AACAPC may be a nasal spray or a throat and mouth spray placed in a vial or bottle with a dispensing valve or with a dropper dispenser.

The medicinal drug may be said AACAPC in a suitable for inhalation form placed in a vial or a vial, a container, or an ampoule for nebulizer inhalation.

Another subject of the invention is the use of an AACAPC with aprotinin activity between 50 KIU/ml and 950 KIU/ml or a drug containing said AACAPC for the treatment and prevention of ARVI.

The aforementioned AACAPC may contain excipients. The AACAPC-based medicinal drug may be in a form suitable for inhalation or for nasal or drip administration. Preferred possible dosage forms are as mentioned above.

AACAPC or an AACAPC-based medicinal drug is most preferably used for the prevention and treatment of ARVI, including in patients infected predominantly with influenza viruses or coronaviruses, including SARS-CoV-2. Also preferred is the use of AACAPC or a drug based thereon for the prevention and treatment of influenza pneumonia or coronavirus COVID-19.

The most preferred device for ARVI treatment and prevention is a bottle with a dispensing valve for nasal or throat and mouth sprays or with a dropper dispenser, said device containing AACAPC with aprotinin activity between 50 KIU/ml and 950 KIU/ml optionally containing excipients, or a drug based thereon according to this invention suitable for inhalation or nasal or drip administration.

A further subject of the present invention is the use for therapeutic and preventive purposes of a drug in the form of a nasal spray or a throat and mouth spray in a device that is a bottle with a dispensing apparatus or with a dropper dispenser containing AACAPC with aprotinin activity from 50 KIU/ml to 950 KIU/ml, optionally containing excipients.

Preferred is the use of the AACAPC of the present invention or a medicinal drug or device based thereupon for the prevention and treatment of ARVI patients.

The study has shown high efficacy for:
AACAPC with a prophylactic and therapeutic aprotinin activity of 950 KIU/ml shown in an inhalation-induced influenza pneumonia model of A/California/04/2009 (H1N1) infected mice;
prophylaxis against SARS-CoV-2 (COVID-19) using a vial with a dosing valve containing an AACAPC nasal spray with aprotinin activity of 400 KIU/ml given to healthcare workers who worked continuously in the “red zone” of a COVID hospital, and the volunteers who took part in the study were free of the side effects indicated above for known drugs;
inhalation (nebulizer) therapy of patients hospitalized with COVID-19 pneumonia of moderate severity using AACAPC with an activity of 62.5 KIU/mL containing aprotinin, wherein patients did not experience the side effects indicated above for known drugs and all were discharged from the hospital.

This invention is illustrated by the following drawings:

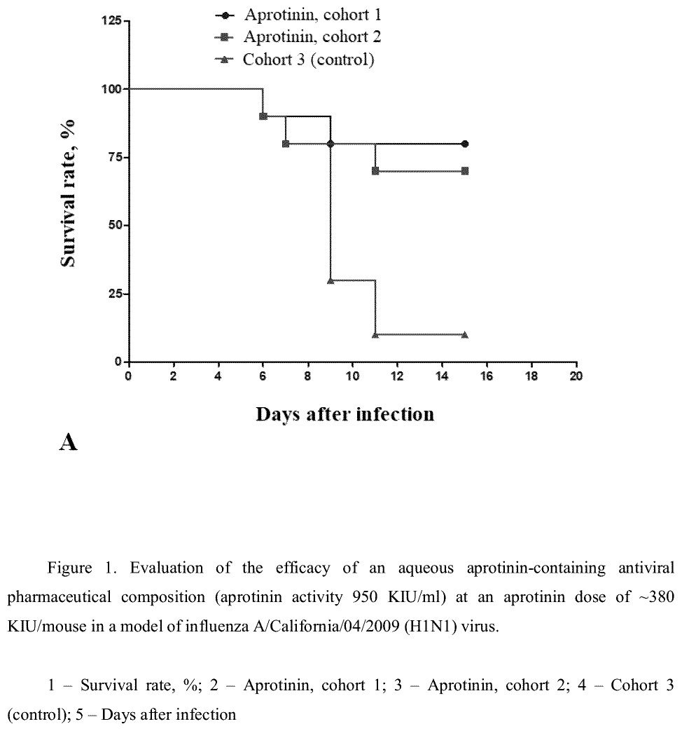

FIG. 1. Evaluation of the efficacy of AACAPC (aprotinin activity 950 KIU/ml) at an aprotinin dose of ˜380 KIU/mouse in a model of influenza A/California/04/2009 (H1N1) virus. A—Assessment of animal survival.

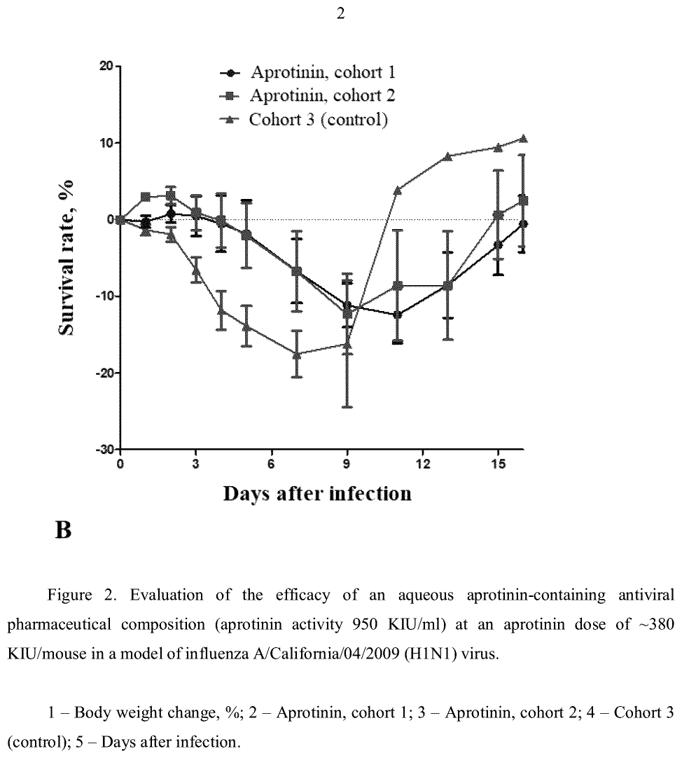

FIG. 2. Evaluation of the efficacy of AACAPC (aprotinin activity 950 KIU/ml) at an aprotinin dose of ˜380 KIU/mouse in a model of influenza A/California/04/2009 (H1N1) virus. B—Change in animal body weight.

This invention is illustrated by, but not limited to the following examples.

Example 1. AACAPC for inhalation and spray therapy and prevention of acute respiratory infections. Sodium chloride (8.5 mg), benzyl alcohol (10 mg), and aprotinin (1.55 mg, 6490 KIU/mg) are dissolved at room temperature in 154 ml of normal saline (0.9% sodium chloride in water) to obtain 155 ml of aqueous AACAPC (aprotinin activity 65 KIU/ml) containing 1.55 mg of aprotinin.

Example 2. AACAPC for inhalation and spray therapy and prevention of acute respiratory infections. Sigma-Aldrich (USA) aprotinin (assay 98%, 1 mg) with an activity of 6500 KIU/mg is dissolved [https://www.sigmaaldrich.com/catalog/search?term=A6103&interface=All&N=0&mode=partialmax&lang=en&region=US&focus=product&F=PR&ST=RS&N3=mode % 20matchpart ialmax&N5=All] in 100 ml of normal saline (0.9% sodium chloride in water) to obtain 100 ml of AACAPC (aprotinin activity 65 KIU/ml) containing 1 mg of aprotinin.

Example 3. AACAPC for inhalation and spray therapy and prevention of acute respiratory infections. The contents of one vial of Aprotex Lyophilzate [https://www.rlsnet.ru/tn_index_id_26457.htm] containing 13 300 KIU of aprotinin, 30 mg of lactose and sodium hydroxide (to adjust to pH 6.0) are dissolved in 100 ml of normal saline to obtain 100 ml of aqueous AACAPC (aprotinin activity 133 KIU/ml).

Example 4. A pharmaceutical composition for inhalation and spray therapy and prevention of acute respiratory infections. Purified Wanhua Biochem (China) aprotinin No. NATE-1892, GMP (assay 95%, 3.703 mg, 5400 KIU/mg) [https://www.creative-enzymes.com/ product/aprotinin-from-bovine-recombinant_16436.html] is dissolved in 100 ml of normal saline to obtain 100 ml of AACAPC with an aprotinin activity of 54 KIU/ml.

Example 5. AACAPC for inhalation and spray therapy and prevention of acute respiratory infections. Gordox® (10 ml) containing aprotinin (15.4 mg, 100 000 KIU) and normal saline (240 ml) are mixed at room temperature to obtain 250 ml of AACAPC (aprotinin activity 400 KIU/ml) containing 15.4 mg of aprotinin.

Example 6. A pharmaceutical composition for inhalation and spray therapy and prevention of acute respiratory infections. Gordox® (9.5 ml) containing aprotinin (15.4 mg, 95 000 KIU) and normal saline (90 ml) are mixed at room temperature to obtain 100 ml of AACAPC (aprotinin activity 950 KIU/ml) containing 15.4 mg of aprotinin.

Example 7. Devices for nasal spray therapy and prevention of acute respiratory infections. AACAPC (400 KIU/ml) as produced in Example 5 is poured into 20 ml or 50 ml medical glass or plastic vials (bottles) with a nasal sprayer [https://aligid.ru/item/32823265496] to obtain devices for nasal spray therapy and prevention of acute respiratory infections.

Example 8. Devices for nasal, drip or spray therapy and prevention of ARVI with an aprotinin activity of 950 KIU/ml as produced in Example 6 are poured into 20 ml or 50 ml glass or plastic spray bottles with a screw-neck and a drip or spray dispenser for nasal administration of drugs [https://www.era-vodoleya.ru/meditsinsKIU/] to obtain devices for nasal drip or spray therapy and prevention of acute respiratory infections.

Example 9. Efficacy of inhalation treatment of influenza pneumonia in mice by AACAPC with an aprotinin activity of 950 KIU/ml as produced in Example 6 in an influenza pneumonia model of A/California/04/2009 (H1N1) infected mice.

The study involved three cohorts of 20 mice (female, Balb/c line) each: 10 mice to assess survival, 5 mice to assess virus titer in the lungs 24 hours after infection, and 5 mice to assess virus titer in the lungs 96 hours after infection.

Group-randomized mice were infected intranasally with A/California/04/ influenza virus under mild anesthesia at a dose of 5 MLD50/ml (25 μl in each nostril—10^{4,5} TCID50/0.1 ml).

Cohort 1 mice were treated with AACAPC with an aprotinin activity of 950 KIU/ml as obtained in Example 6 according to prophylactic regimen 1: on day 0, 3 inhalations were given (in the morning, in the afternoon, and in the evening); on day 1, inhalation was given in the morning and one hour later the animals were infected with influenza virus; then, inhalations were given in the afternoon and in the evening followed by 3 inhalations for 10 days.

Cohort 2 mice were treated with AACAPC with an aprotinin activity of 950 KIU/ml as obtained in Example 6 according to therapeutic regimen 2: on day 1, inhalation was given in the morning and 1 hour later the animals were infected with influenza virus; then, inhalations were given in the afternoon and in the evening, followed by 3 inhalations for 10 days. The mice of cohort 3—the viral control cohort—received no treatment; on day 1, they were infected with influenza virus in the morning, and distilled water was given intragastrically immediately after infection in the evening of the same day.

AACAPC with an aprotinin activity of 950 KIU/ml as obtained in Example 6 was inhaled using a nebulizer. For this purpose, a group of 5 animals was placed in a box wherein they inhaled 2 ml of the test drug for 10 min. This corresponds to an aprotinin dose of ˜380 KIU/mouse.

Inhalation treatment of mice with AACAPC having aprotinin activity of 950 KIU/ml as obtained in Example 6 at an aprotinin dose of ˜380 KIU/mouse, 3 inhalations daily, for 10days, according to the prophylactic and therapeutic regimen, effectively protected 60-80% of animals from death and reduced their weight loss, increased their average life expectancy, and significantly inhibited virus multiplication in the lungs of animals compared to the viral control group by an average of 3 log TCID₅₀/0.1 ml (Table 1, FIGS. 1 and 2).

TABLE 1
Efficacy of AACAPC with an aprotinin activity of 950 KIU/ml at an aprotinin
dose of ~380 KIU/mouse in a mouse model of influenza pneumonia when
infected with mouse-adapted A/California/04/2009 (H1N1) influenza virus

| | Survival rate | | Average life | Viral titer, log TCID50/0.1 ml^{b} | |
|---|---|---|---|---|---|
| | | Death | expectancy | 24 h after | 96 h after |
| Cohorts | Alive/total | rate, % | (days) | infection | infection |
| Cohort 1 | 8/10 | 20 | 13.4 (1-7 d, | 2.9 ± 2.66 (5; | 2.7 ± 2.49 (4; |
| | (p = 0.000538)^{a} | | 1-9 d) | 0; 0; 4.5; 5)^{c} | 0; 0; 5; 4.5) |
| Cohort 2 | 7/10 | 30 | 12.6 (1-6 d, | 3 ± 2.74 (5; | 3.1 ± 2.84 (5; |
| | (p = 0.004103) | | 1-7 d, 1-11 d) | 0; 0; 5; 5) | 0; 0; 5.5; 5) |
| Cohort 3 | 1/10 | 90 | 8.6 (1-6 d, | 6.2 ± 0.910 (6.5; | 5.6 ± 0.82 (5; |
| | | | 1-7 d, 5-9 d, | 6; 5; 7.5; 6) | 5; 5; 6.5; 6.5) |
| | | | 2-11 d) | | |

^{a}p < 0.05 is considered a reliable difference from control.
^{b}TCID50—tissue cytopathic infectious dose 50.
^{c}Decrease in titer on average >2 log EID50 is considered a significant difference from the control group.

Example 10. Preventing SARS-CoV-2 in health care workers who constantly worked in the COVID-19 hospital red zone by using a nasal spray device containing AACAPC with an aprotinin activity of 400 KIU/ml as per Example 7.

Thirty-two health care workers, including medical and nursing staff, who worked continuously in the red zone during the three-month follow-up took part in the study. Among them: 5 intensive care physicians, 2 surgeons who performed tracheostomies and pleural punctures, 15 physicians, and 10 nurses. Three weeks after the opening of the COVID hospital, the medical workers who were constantly working in the red zone along with supportive therapy began to use the nasal spray device from Example 7 containing AACAPC as obtained in Example 5, with an aprotinin activity of 400 KIU/ml, twice daily: in the morning, before entering the red zone, and in the evening, after exiting the red zone. All study participants were tested weekly for the presence of SARS-CoV-2 using polymerase chain reaction (PCR). On week 6, all study participants were tested for specific IgG. It was found that the study participants had no adverse effects and only 2 (6.7%) of 30 were infected and had mild or no symptoms at all. At the same time, according to published data [Dawei Wang et al. JAMA 2020, 323(11): 1061-1069. https://jamanetwork.com/journals/jama/fullarticle/2761044], the infection rate of medical personnel who constantly worked in the red zone without aprotinin prophylaxis reached 29% (4.3 times higher).

Example 11. Inhalation treatment of patients hospitalized with COVID-19 pneumonia of moderate severity by AACAPC from Example 1 with an aprotinin activity of 65 KIU/ml. Ten patients (Table 2) who received AACAPC as obtained in Example 1 (aprotinin activity 65 KIU/ml) by inhalation in addition to standard therapy were included in the clinical trial. The patients inhaled AACAPC via an Omron NE-C300 Complete nebulizer: dosing mode 3 (lower respiratory tract), particle size 3 microns, nebulization rate 0.3 ml/min, aprotinin dose with an activity of 625 KIU per inhalation (9.6 ml AACAPC as obtained in Example 1), 4 inhalations daily for 5 days.

TABLE 2
Demographic and other baseline characteristics of patients
hospitalized with moderate COVID-19 pneumonia

| Status | Characteristics | |
|---|---|---|
| ¹Age (years) | 48.2 ± 10.4 | |
| 18-44 | 4 | (40.0%) |
| 45-59 | 3 | (30.0%) |
| ≥60 | 3 | (30.0%) |
| Sex | | |
| Male | 1 | (10.0%) |
| Female | 9 | (90.0%) |
| ¹ Body weight (kg) | 76.7 ± 6.4 | |
| BMI (kg/m²) | 26.9 ± 2.1 | |
| ¹ Duration of illness (days) | 2.7 ± 0.7 | |
| ≤7 days | 10 | (100.0%) |
| >7 days | 0 | (0.0%) |
| Ordinal scale | | |
| 3 | 0 | (0.0%) |
| 4 | 10 | (100.0%) |
| ¹SpO2 (%) | 94.3 ± 0.7 | |
| ≥95 | 6 | (60.0%) |
| <95 | 4 | (40.0%) |
| Temperature (° C.) | 38.3 ± 0.3 | |
| <37 | 0 | (0.0%) |
| 37-38 | 1 | (10.0%) |
| >38 | 9 | (90.0%) |
| ¹RR (per minute) | 22.6 ± 0.7 | |
| ≤22 | 5 | (50.0%) |
| >22 | 5 | (50.0%) |
| ² CRP (mg/1) | 40.5 | (32.0-45.8) |
| Norm | 0 | (0.0%) |
| Elevated | 10 | (100.0%) |
| ¹D-dimer (ng/ml) | 820.1 ± 133.1 | |
| Norm | 0 | (0.0%) |
| Elevated | 10 | (100.0%) |
| ²Involvement of lung parenchyma (%) | 18.5 | (16.3-24.5) |
| Cohort 1 | 3 | (30.0%) |
| Cohort 2 | 7 | (70.0%) |
| Cohort 3 | 0 | (0.0%) |

¹mean ± SD,
²median (Q1-Q3).

The clinical trial has shown high efficacy of AACAPC of this invention in the inhalation therapy of patients hospitalized with moderately severe COVID-19 pneumonia. Thus, the median time to first negative PCR result with a confirmed consistent second negative result was 9.0 (5.0-9.0) days; the median time to normalization of CRP (≤10 mg/l) was 4.0 (3.0-5.0) days; the median time to normalization of D-dimer (<243 ng/ml) was 9.0 (5.0-9.0) days; the median time to normalization of body temperature (<37° C.) was 4.5 (3.0-5.0) days; and the median time to improvement of clinical status by 2 points on an ordinal scale was 6.0 (6.0-6.0) days. No patients were transferred to the ICU for ALV or NIV. Computed tomography revealed that on day 14, 10 (100.0%) patients showed improvement relative to the baseline. No adverse events or side effects were reported in the patients during the study. All patients were discharged from the hospital.

1. An aqueous aprotinin-containing antiviral pharmaceutical composition (AACAPC) with aprotinin activity from 50 KIU/ml to 950 KIU/ml for the prevention and treatment of acute respiratory viral infections (ARVI).

2. The composition according to claim 1 containing excipients.

3. The composition according to claim 1 or 2 containing as excipients sodium chloride and benzyl alcohol or sodium hydroxide and lactose.

4. A medicinal drug for the prevention and treatment of ARVI comprising an aqueous aprotinin-containing antiviral pharmaceutical composition (AACAPC) with aprotinin activity from 50 KIU/ml to 950 KIU/ml according to any of claims 1 to 3 in a form suitable for inhalation, nasal, or drip use.

5. The medicinal drug according to claim 4 containing AACAPC according to any of claims 1 to 3, which is a nasal spray or a throat and mouth spray placed in a bottle or in a vial with a dosing valve or in a bottle with a dropper dispenser.

6. The medicinal drug according to claim 4 containing AACAPC according to any of claims 1 to 3 in a form suitable for inhalation placed in a bottle or in a vial, container, or ampoule for nebulizer inhalation.

7. Use of an aqueous aprotinin-containing antiviral pharmaceutical composition (AACAPC) with aprotinin activity from 50 KIU/ml to 950 KIU/ml according to any of claims 1 to 3, or a medicinal drug containing thereof according to any of claims 4 to 6 for the prevention and treatment of ARVI.

8. The use according to claim 7 for the prevention and treatment of ARVI in patients infected with influenza viruses or coronaviruses.

9. The use according to claim 7 or 8 for the prevention and treatment of ARVI in patients infected with SARS-CoV-2.

10. The use according to any of claims 7 to 9 for the prevention and treatment of influenza pneumonia.

11. The use according to any of claims 7 to 9 for the prevention and treatment of coronavirus disease COVID-19.

12. The use according to any of claims 7 to 11 of a medicinal drug for ARVI prevention and treatment in the form of a nasal spray or a throat and mouth spray placed in a device, which is a vial with a dosing valve or with a dropper dispenser containing AACAPC with aprotinin activity from 50 KIU/ml to 950 KIU/ml according to any of claims 1 to 3.

13. The use according to claim 12 wherein the pharmaceutical composition contains excipients.

14. A device for the prevention and treatment of ARVI, which is a vial with a dosing valve for a nasal spray or a throat and mouth spray, or with a dropper dispenser containing AACAPC with aprotinin activity from 50 KIU/ml to 950 KIU/ml according to any of claims 1 to 3, or a medicinal drug based thereon suitable for inhalation, nasal, or drip use according to claim 4.
